(19) United States
(12) Patent Application Publication
(10) Pub. No.: US 2011/0129843 A1
(43) Pub. Date: Jun. 2, 2011

(54) METHOD FOR EVALUATING THE VIRULENCE OF PATHOGENIC BIPHASIC BACTERIA

(76) Inventors: Scott Martell Boyette, New Hope, PA (US); Jing Chen, Shanghai (CN); Jie Li, Shanghai (CN); Weiqing Xu, Shanghai (CN); Kechao Yang, Shanghai (CN)

(21) Appl. No.: 13/054,988

(22) PCT Filed: Jul. 29, 2009
(86) PCT No.: PCT/US2009/052055

§ 371 Date: Jan. 20, 2011

Related U.S. Application Data
(60) Provisional application No. 61/084,905, filed on Jul. 30, 2008.

(51) Int. Cl. C12Q 1/68

(52) U.S. Cl. 435/6.12; 435/6.15

(57) ABSTRACT

A method for evaluating relative bacterial virulence of a biphasic bacteria in environmental systems includes measuring the concentration of DNA in the bacteria, measuring the concentration of RNA in the bacteria, determining a ratio of the concentration of RNA to the concentration of DNA and correlating the concentration ratio with a level of relative pathogenicity, wherein the bacteria is preferentially Legionella pneumophila, Mycobacterium tuberculosis and Listeria.

### CROSS-REFERENCE TO RELATED APPLICATIONS

This patent application claims priority to a PCT patent application filed on Jul. 29, 2009 application number PCT/US09/52055 which claims the benefit of the U.S. Provisional Patent Application No. 61/084905 filed on Jul. 30, 2008.

### FIELD OF THE INVENTION

The present invention is related to a method for measuring pathogenic biphasic bacteria in environmental systems and. more particularly, for evaluating the virulence of pathogenic triphasic bacteria in environmental systems.

### BACKGROUND OF THE INVENTION

The presence of pathogenic bacteria in environmental or clinical samples for water, food, healthcare or pharmaceutical businesses can raise serious health concerns. Evaluating the pathogenic bacteria to determine its virulence is critical to assessing the relative risk of these samples. Conventional assays, such as culture-based methods or hybridization-based methods, can be used to test the concentration of microbial pathogens. However, culture-based methods require lengthy incubation time and the method is susceptible to producing false results, because field samples can interfere with the method. Also, it is difficult to accurately detect low levels of pathogenic bacteria with hybridization-based methods. More importantly, output for both methods is only the bacteria concentration, not pathogenic virulence, which is of greater concern to the public and business community. Accordingly, a need exists for an improved method and system for measuring the relative virulence of biphasic pathogenic bacteria that is fast and accurate and provides low levels of detection.

### SUMMARY OF THE INVENTION

In one embodiment, a method for evaluating relative pathogenic virulence of a biphasic bacteria in environmental systems including measuring the concentration of DNA in the bacteria, measuring the concentration of RNA in the bacteria, determining a ratio of the concentration of RNA to the concentration of DNA and correlating the concentration ratio with a level of relative pathogenicity.

The various embodiments provide a quick, accurate and cost-effective method for detecting and measuring the relative virulence of biphasic pathogenic bacteria at early onset while the pathogens are at low concentrations.

### BRIEF DESCRIPTION OF DRAWINGS

FIG. 1 is a graph showing the plate count for Legionella pneumophila. The graph is the log of CFU/ml vs. time in hours.

FIG. 2 is a graph showing the DNA copies for Legionella pneumophila as measured by real-time PCR. The graph is the log of DNA (GU) vs. time in hours. FIG. 3 is a graph showing the rRNA copies for Legionella pneumophila as measured by real-time TMA. The graph is the log of rRNA copies vs. time in hours.

FIG. 4 is a graph showing the rRNA/DNA ratio for Legionella pneumophila. The graph is the log of rRNA/DNA ratio vs. the phase of the Legionella pneumophila (Lpn phase).

### DETAILED DESCRIPTION OF THE INVENTION

The singular forms “a,” “an” and “the” include plural referents unless the context clearly dictates otherwise. The endpoints of all ranges reciting the same characteristic are independently combinable and inclusive of the recited endpoint. All references are incorporated herein by reference. The modifier “about” used in connection with a quantity is inclusive of the slated value and has the meaning dictated by the context (e.g., includes the tolerance ranges associated with measurement of the particular quantity).

“Optional” or “optionally” means that the subsequently described event or circumstance may or may not occur, or that the subsequently identified material may or may not be present, and that the description includes instances where the event or circumstance occurs or where the material is present, and instances where the event or circumstance does not occur or the material is not present.

In one embodiment, a method for evaluating relative pathogenic virulence of a biphasic bacteria in environmental systems including measuring the concentration of DNA in the bacteria, measuring the concentration of RNA in the bacteria, determining a ratio of the concentration of RNA to the concentration of DNA and correlating the concentration ratio with a level of relative pathogenicity.

Pathogenic biphasic bacteria in environmental systems can create health problems. These pathogens have developed specific strategies for coping with different environmental stress conditions. The bacteria pass through four different phases. The initial phase is a lag phase in which the bacteria are maturing, but cannot divide. The exponential phase is where the cells multiply. Upon entry of a host cell, gene expression will be altered to permit multiplication. The bacteria remains in the exponential phase while there are plenty of nutrients in the environment When the nutrients become limited or start to become scarce, the bacteria begin to transform into a stationary phase (also known as post-exponential phase) in which the rate of growth is near or equal to the rate of death. During the stationary phase, the pathogens switch metabolisms to enhance infectivily. Upon entry of a host cell, gene expression will be altered to permit multiplication. The stationary phase is the most virulent phase, because it allows the bacteria to enhance infection. Following the stationary phase, is the dead phase in which the nutrients are depleted and the bacteria die. The bacteria population may be a a single species at a single growth phase or a mixed population at different growth phases, or any combination of the the four phases. These four phases are also observed in laboratory-grown cultures.

Biphasic pathogenic bacteria are any type of pathogen that can shift its metabolic processes and after its cellular expressions and extracellular activities to allow the pathogen to seek a host that can provide essential growth conditions for replication. In one embodiment, biphasic pathogenic bacteria include, but are not limited to, Legionella pneumophila, Mycobacterium tuberculosis or Lysteria.

The enviromental systems may be any type of environment where biphasic pathogenic bacteria can invade. In one embodiment, the environmental systems may be liquid, solid or air. In one embodiment, the enironmental system may be soil, aerosolized fluids containing host cells that can harbor pathogenic bacteria or aqueous media. In one embodiment, the aqueous media may be water, blood, urine, sputum, bodily fluids or any combination of the foregoing. In another embodiment, the liquid media may be cooling tower water, wastewater or other industrial fluid processes from water, food, healthcare or pharmaceutical businesses. The concentration of DNA for the biphasic bacteria may be measured in any suitable manner. In one embodiment, the DNA concentration may be measured by real-time polymerase chain reaction (PCR) on DNA extracted from the biphasic bacteria. In another embodiment, the DNA concentration is measured by real-time PCR using macrophage infectivity potentiator (mip) gene targeting primers, probes and thermal-stable enzymes on DNA extracted from the biphasic bacteria.

The primers and thermal stable enzymes are used to amplify the DNA exponentially for measuring. The primers are short DNA fragments, which match the DNA to be measured, and the thermal-stable enzyme assembles the primers into new DNA strands. The thermal-stable enzyme may be a Taq polymerase, such as a Taqman* probe.

The probe contains a DNA template and a fluorescent marker. The DNA template is a specific DNA sequence on a substrate, which allows the probe to only target or measure DNA matching the DNA template. The fluorescent marker attaches to the DNA to monitor the amplified DNA. The fluorescence marker may be any type of fluorescent dye or indicator that changes its fluorescence signal in the presence of DNA. In one embodiment, the fluorescent dye is a fluorochrome or fluorophore, which are microbiological staining dye that bind with nucleic acids, in one embodiment, the fluorophore may be 5-carboxytetramethylrhodamine (TAMRA).

Fluorescence may be measured by any type of fluorescence detector. In one embodiment, the fluorescent signal is measured by fluorescence spectroscopy, fluorescence microscopy, fluorescence diode array detection, micro plate fluorescence reading or flow cytometry.

The concentration of RNA for the Diphasic bacteria may be measured in any suitable manner. The selected RNA can be either messenger RNA (mRNA) or ribosomal RNA (rRNA). In one embodiment, the RNA may be extracted from the biphasic bacteria and measured by methods including, but not limited to, Northern blotting, ribonuclease protection assays, in situ hydridization, real-time Transcription Mediated Amplification (TMA) or reverse transcriptase polymerase chain reaction.

hybridization probe complementary to at least a part of the target RNA sequence to detect me RNA. The hybrid signals are detected by X-ray film and quantified by densitometry. In situ hybridization uses a labeled probe containing a complementary RNA strand to detect the target RNA. The RNA may be quantified by measuring fluorescence, radiography or immunohistochemistry. In reverse transcription polymerase chain reaction, the RNA strand is reverse transcribed into its DNA complement using an enzyme reverse transcriptase and the resulting complementary DNA is amplified and measured using real-time PCR as described above. The TMA is a nucleic acid amplification test, which is commercially available from Gen-Probe, Inc.

The nucleic acid (DNA and RNA) from the biphasic bacteria cells may be extracted by any suitable manner, in one embodiment, the nucleic acid from the pathogenic cells may be extracted by lysing the cells. Lysing may be performed using mechanical, chemical, physical, electrical, ultrasonic or microwave methods or any combination of these methods.

Mechanical lysing physically disrupts the cell barriers, such as by shear, vibration or force. Examples of mechanical methods include, but are not limited to, pressure-driven cell flow through fiiter-like structures or small scale bars in fluidic channels, osmotically stressing cells with rapid diffusional mixing of low ionic-strength water, subjecting cells to shear forces while entering a special region with sharp small-scale structures, disrupting cell barriers with a minibead beater or bead mill or applying ultrasonic energy to the cells in the aqueous medium.

Chemical lysing occurs when chemicals are used to disrupt the cell barriers and allow the intracellular content to be released Any chemical may be used that can disrupt the cell barriers. In one embodiment, detergents, enzymes, extraction solvents or lysing buffers are used. Detergents include, but are not limited to, dodecyl sulfate, 3-[(3 -cholamidopropyl)diinethylammonio]-1-propanesulfonate, TWEEN™ 20 detergent, TRITON™ X series detergents, sodium etiolate, sodium deoxycholate, guanidinium chloride. Enzymes include, but are not limited to, lysozymes, mutanolysin, labiase. lysostaphin, lyticase, proteinase K, endolysin or achromopeptidases. Extraction solvents include, but are not limited to, poly vinylpolypvrrolidone, phenol, trichlorotrifluoroelhane or a mixture of phenol and guanidinium thiocyanate or guanidinium chloride. Lysing buffers include, but are not limited to, ammonium chloride, quaternary ammonium compounds, hexadecyltrimethylammonium bromide, cetyltrimethylammonium bromide, sodium dodecyl sulfate, hexametaphosphate, sodium pyrophosphate, Swab Transfer Medium (STM), a lysing solution available commercially from Gen-Probe, Inc., Zap-o-globin™, a lysing buffer available commercially from Coulter Diagnostics or CyQUANT™ cell lysis buffer, available commercially from Molecular Probes.

The reagent may be added in any amount suitable for lysing the microbiological matter and may be added in excess. In one embodiment, the reagent is added in an amount of from about 1 ml to about 10,000 ml per milliliter of aqueous medium. In another embodiment, the reagent is added in an amount of from about 1 ml to about 1000 ml per milliliter of aqueous medium. In another embodiment, the reagent is added in an amount of from about 1 ml to about 50 ml per milliliter of aqueous medium.

Physical lysing may occur thermally or by freeze-thawing. Cell lysing can be accomplished thermally by heating the aqueous medium, such as with a thermal block or hot plate, in one embodiment, the aqueous medium is heated to a temperature from about 40° C. to about 100° C. in another embodiment, the temperature is from about 40° C. lo about 60° C. to one embodiment, the aqueous medium is heated from about 1 minute to about 1 hour. In another embodiment, the aqueous medium is heated from about 1 minute to about 30 minutes, including from about 1 minute to about 15 minutes, In another embodiment, the aqueous medium is heated from about 1 minute to about 3 minutes. In one example of freeze-thawing, the aqueous medium is frozen, such as in an ethanol-dry ice bath, and then thawed.

Cells may be lysed electrically with a series of electrical pulses, by diffusive mixing and dielectrophoretic trapping or by microwave radiation. Free radicals may also be used for cell lysing. The method includes applying an electric field to a mixture of a metal ion, peroxide and the microbiological matter in the aqueous medium to generate free radicals, which attack the cell barriers.

In one embodiment, the nucleic acids extracted from the cell lysate may be purified to obtain the specific target DNA and specific target RNA. In one embodiment, the nucleic acids may be purified by chemical precipitation and dissolution, magnetic beads or affinity to resin through non-specific adsorption or by attachment to complementary primers, in one embodiment, during chemical precipitation, solvents may be added to the cell lysate to prepare a solution and precipitation solvents may be mixed with the extracted nucleic acids to precipitate out the specific target nucleic acids and remove impurities with the solvents. In one embodiment, the precipitation solvents include, but are not limited to, ethanol and isopropanol. During dissolution, a dissolution solvent is added to redissolve the nucleic acids after precipitation. Water soluble impurities have limited solubility in me dissolution solvents and do not redissolve. Dissolution solvents may include lithium chloride, guanidium chloride or the combination of an alcohol with a monovalent cation.

In another embodiment, nucleic acids may be purified by magnetic beads through a bind-wash-elute procedure, in one embodiment, the magnetic beads may be Promega* MagneSil* Red, which is commercially available from the Promega Corporation or Seradyn* bead, which is commercially available from Seradyn Inc.

lh the affinity to resin with complementary primers method, DNA templates are used to select the target DNA. The DNA template is a complementary oligonucleotide sequence on a substrate.

In one embodiment, the purification of the extracted nucleic acids can be automated. In another embodiment, the purification is automated by using a

KingFisher® instrument available commercially from Thermo Electron Corporation.

The ratio of the concentration of RNA to the concentration of DNA is determined. The ratio indicates the probability that the triphasic bacteria exist in a specific growth phase and provides a parameter for evaluating the relative virulence of the pathogenic bacteria. The triphasic bacteria contain cells in the lag phase, the exponential growth phase, in which the cells resemble intracellular cells that are altering to permit multiplication, and the post-exponential phase in which the cells resemble extracellular cells and possess increased virulence.

The ratio of the concentration of RNA to DNA may be equated with a level of relative pathogenicity. In one embodiment, the ratio is equated with a level of relative pathogenicity by comparing the ratio against a reference curve. In one embodiment, a reference curve may be prepared for each pathogen of interest. In another embodiment, a reference curve is prepared by monitoring the concentration of DNA and RNA through different growth phases. In one embodiment, culture-based plate count methods are used to determine the growth phases of the pathogen.

In order that those skilled in the art will be better able to practice the present disclosure, the following examples are given by way of illustration and not by way of limitation.

### EXAMPLES

### Example 1

Preparation of a reference curve for determining the virulence of Legionella pneumophila.

3-5 Legionella pneumophila colonies were removed from a previously populated culture media plate and grown in a liquid culture media for 48-72 hours and added to 40 ml of fresh sterilized liquid media to form a sample. The sample was shaken (175 rpm) at 36° C. for 24 hrs.

The Legionella pneumophila sample was added to another fresh sterilized liquid media in a 1:40 volume ratio to prepare a reference sample. The sample was shaken (175 rpm) at 36° C. for 24 hrs.

The reference sample was tested to determine the stage of the Legionella pneumophila and the concentrations of DNA and RNA at various time points: 1.5 hr (as lag phase), 6 hr, 9 hr (as exponential phase), 26 hr, 28 hr, 30 hr, 32 hr, 34 hr, 48 hr, 51.5 hr, 73.5 hr and 77 hr (as post-exponential phase).

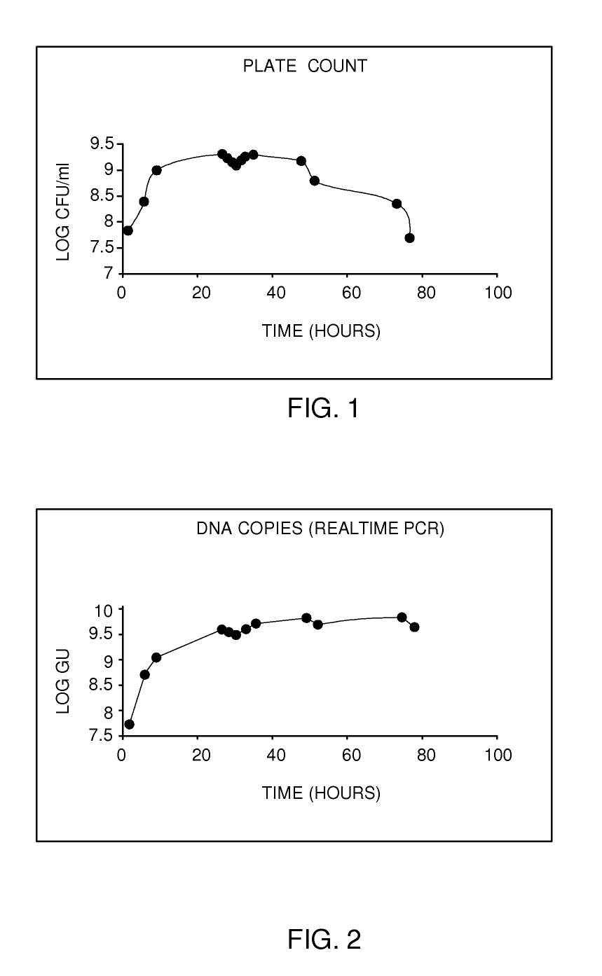

Plate count tests were performed at each time point to measure the growth phase of the Legionella pneumophila. Standard plate count methods in accordance with testing standards AFNOR 90-431 or ISO 11731 were used. Three replicates were performed at each time point and the results were the average of the three replicates. The plate count tests look about 10 days to complete and the data are shown in FIG. 1.

Real-time PCR and real-time Transcription Mediated Amplification (TMA) tests were performed at each time to measure the concentration of the DNA and RNA of the Legionella pneumophila, respectively. Initially, the nuclear material was extracted from the Legionella pneumophila. 1 ml of the initial sample at each time was removed and spun down in a centrifuge at 3000 g for 2 min. The supernatant was removed and disposed. 1 ml of sterile page's saline (0.012% (w/v) sodium chloride, 0.0004% (w/v) magnesium sulfate pentahydrate, 0.0004% (w/v) calcium chloride dehydrate, 0.0.142% (w/v) disodium hydrogen phosphate, 0.0136% (w/v) potassium dihydrogen phosphate (136 mg/L)) was added to re-suspend the sample. 100 μl of the re-suspended sample was removed and lysed with 3 ml of a chemical lysis buffer, STM, for at least 3 hrs. The Real-time PCR test used a bead-based DNA purification method. 500 μl of the lysate was purified with Promega* MagneSil* Red (available commercially from Promega Corporation). The primers (mip6 and mip8) amplified a 110-bp fragment of the mip gene, and the amplification was detected with a TaqMan* probe TO-mip (Labeled with 5′-FAM/3′-TAMRA). Data is shown in FIG. 2. The Real-time TMA test was a transcription-based method to detect RNA.

500 μl of the lysate was purified with Seradyn* bead and a region of the Legionella Pneumophila 23S rRNA was amplified. The amplification product was detected with a torch probe labeled with a 5-carboxytetramethylrhodamine (TAMRA) fluorophore. Data is shown in FIG. 3.

Data analysis was performed after getting all results. rRNA/DNA ratio=rRNA copies determined with TMA/DNA genomic units (GU) determined with real time PCR rRNA copies/CFU=rRNA copies determined with TMA)/colony forming units (CFU) determined by the plate count method

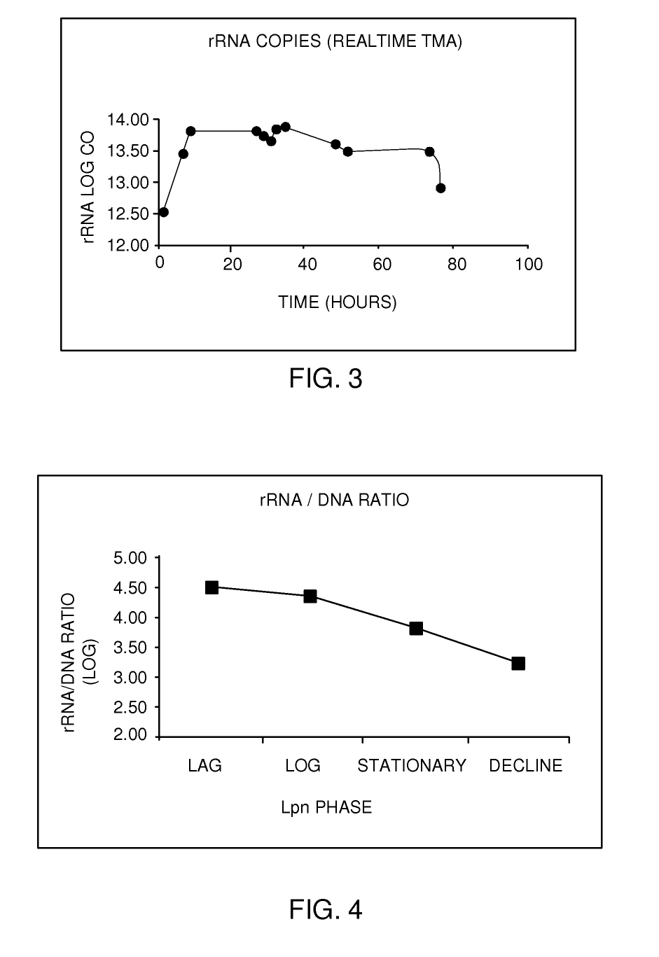

The average RNA/DNA ratio for the exponential phase was 22,542 and the average for the stationary phase was 6685. A reference curve was prepared with this data and is shown in FIG. 4.

The target RNA/DNA ratio based method identified the specific triphasic pathogen growth phase and evaluated its relative virulence in less than 3 hours.

### Example 2

Planktonic Legionella pneumophila cells were obtained from various 50 ml cooling tower water samples through filtration-based concentration. The samples were filtered through a polyethersulfone (PES) 0.45 μm membrane. The cells were lysed on the membrane with 3 ml of a chemical lysis buffer, STM, overnight and the lysates were filtered through a PES 0.22 μm membrane to remove the cell debris.

DNA and rRNA in the lysates were quantified according to the methods described in Example 1.

As shown in Table 1, the majority of the rRNA/DNA ratio from these field samples resides in the range of 300 to 9000, which indicates the growth phase.

TABLE 1

| Sample | 1 | 2 | 3 | 4 | 5 | 6 | 7 | 8 | 9 |
|---|---|---|---|---|---|---|---|---|---|
| rRNA/ | 470 | 1710 | 2898 | 3203 | 14,156 | 4061 | 1221 | 255 | 25,202 |
| DNA | | | | | | | | | |

| Sample | 10 | 11 | 12 | 13 | 14 | 15 | 16 | 17 |
|---|---|---|---|---|---|---|---|---|
| rRNA/ | 2457 | 1788 | 3209 | 3394 | 28,210 | 758 | 3271 | 9474 |
| DNA | | | | | | | | |

Samples 5, 9 and 14 had high RNA concentrations indicating that they may be in a less virulent exponential growth phase, which can result when hosts first emit the bacteria While typical embodiments have been set forth for the purpose of illustration, the foregoing descriptions should not he deemed to be a limitation on the scope herein. Accordingly, various modifications, adaptations and alternatives may occur to one skilled in the art without departing from the spirit and scope herein.

Having described the invention that which is claimed is:

1. A method for evaluating relative bacterial virulence of a biphasic bacteria in environmental systems comprising measuring the concentration of DNA in the bacteria, measuring the concentration of RNA in the bacteria, determining a ratio of
RNA to DNA with a level of relative pathogenicity.

2. The method of claim 1, wherein the biphasic pathogenic bacteria are selected from the group consisting of Legionella pneumophila, Mycobacterium tuberculosis and Lysteria

3. The method of claim 1, wherein the environmental system is liquid, solid or air.

4. The method of claim 3, wherein the enironmental system is selected from the group consisting of soil, aerosolized fluids and aqueous media.

5. The method of claim 4, wherein the aqueous media is selected from the group consisting of water, wastewater, blood, urine, sputum, bodily fluids and any combination of the foregoing.

6. The method of claim 1, wherein the concentration of DNA is measured by real-time polymerase chain reaction on DNA extracted from the biphasic bacteria.

7. The method of claim 6, wherein the real-time polymerase chain reaction uses macrophage infectivity potentiator (mip) gene targeting primers, probes and thermal-stable enzymes.

8. The method of claim 7, wherein the probe contains a DNA template and a fluorescent marker.

9. The method of claim 8, wherein the fluorescent marker is a fluorochrome or fluorophore.

10. The method of claim 8, wherein a fluorescent signal from the fluorescent marker is measured by a fluorescence detection selected from the group consisting of fluorescence spectroscopy, fluorescence microscopy, fluorescence diode array detection, micro plate fluorescence reading and flow cytometry.

11. The method of claim 1, wherein the concentration of RNA is measured by a method selected from the group consisting of Northern blotting, ribonuclease protection assay, in situ hybridization, real-time Transcription Mediated Amplification and reverse transcriptase polymerase chain reaction on RNA extracted from the triphasic bacteria.

12. The method of claim 6, wherein the DNA is extracted from the biphasic bacteria by lysing the cells.

13. The method of claim 12, wherein the cells are lysed by a lysing procedure selected from the group consisting of mechanical, chemical physical, electrical ultrasonic, microwave methods and any combination of the foregoing.

14. The method of claim 13, wherein the extracted DNA is purified to obtain the specific target DNA.

15. The method of claim 14, wherein the extracted DNA is purified by a process selected from the group consisting of chemical precipitation and dissolution, magnetic beads and affinity to resin.

16. The method of claim 11, wherein the RNA is extracted from the biphasic bacteria by lysing the cells.

17. The method of claim 16, wherein the cells are lysed by a lysing procedure selected from the group consisting of mechanical, chemical, physical, electrical, ultrasonic, microwave methods and any combination of the foregoing.

18. The method of claim 11, wherein the extracted RNA is purified to obtain the specific target RNA.

19. The method of claim 18, wherein the extracted RNA is purified by a process selected from the group consisting of chemical precipitation and dissolution, magnetic beads and affinity to resin.

20. The method of claim 1, wherein the ratio is equated with a level of relative pathogenicity by comparing the ratio against a reference curve.

21. The method of claim 20, wherein the reference curve is prepared by monitoring the concentration of DNA and RNA through different growth phases with a culture-based plate count method.
